(19) United States
(12) Patent Application Publication
(10) Pub. No.: US 2010/0151571 A1
(43) Pub. Date: Jun. 17, 2010

(54) CENTIMETER-SCALE, INTEGRATED DIAGNOSTICS INCUBATOR FOR BIOLOGICAL CULTURING

(75) Inventors: Jelena Vukasinovic, Atlanta, GA (US); Ari Glezer, Atlanta, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: GEORGIA TECH RESEARCH CORPORATION, Atlanta, GA (US)

(21) Appl. No.: 12/529,881

(22) PCT Filed: Jul. 3, 2007
(86) PCT No.: PCT/US2007/072777

§ 371 Date: Feb. 26, 2010

Related U.S. Application Data
(63) Continuation-in-part of application No. 11/483,126, filed on Jul. 7, 2006.
(60) Provisional application No. 60/851,222, filed on Oct. 12, 2006.

(51) Int. Cl. C12N 5/07; C12M 3/00

(52) U.S. Cl. 435/374; 435/289.1; 435/303.1

(57) ABSTRACT

Disclosed are portable, disposable, centimeter-scale, integrated diagnostics incubators for use in biological culturing. An exemplary incubator comprises an optically accessible enclosure having a plurality of fluidic ports. A heating element is disposed within the enclosure that is coupled to an external heater controller. An autoclavable microfluidic perfusion chamber is disposed within the enclosure that comprises a cell culture life support chamber, an inlet port disposed in the perfusion chamber, a collection chamber in communication with the culture chamber, an outlet port coupled to the collection pool, and a perfusing substrate. An optically transparent, gas permeable membrane is attachable to the top of the perfusion chamber. The incubators have optical accessibility, forced flow fluidic control, temperature control, are portable and modular, and are inexpensively manufactured. The incubators permit in-the-field drug testing and culturing of biological tissues.

### CROSS REFERENCE TO RELATED APPLICATIONS

This application claim is a continuation-in-part of, and claims priority to co-pending U.S. patent application Ser. No. 11/483,126 entitled “Centimeter-Scale, Integrated Diagnostics Incubator for Biological Culturing” filed on Jul. 7, 2006 which is incorporated herein by reference. This application also claims priority to U.S. Provisional Patent Application No. 60/851,222 entitled “Centimeter-Scale, Integrated Diagnostics Incubator for Biological Culturing” filed on Oct. 12, 2006, which is incorporated herein by reference.

### STATEMENT REGARDING FEDERALLY SPONSORED RESEARCH OR DEVELOPMENT

This invention was made in part with government support under Grant Number I ROI EB00786-01 awarded by the National Institutes of Health. Therefore, the government may have certain rights in this invention.

### BACKGROUND

The present invention relates generally to portable, diagnostics, pocket-size incubators with integrated perfusion, and more particularly, to a biocompatible, disposable, centimeter-scale, incubator workstation 10 for in situ monitoring, handling and scientific studies of biological cultures in the field and within major laboratories.

Currently, the handling of biological samples intended for experimentation and diagnostic analysis is often limited by the need to carefully manage the conditions of perfusion and incubation, and to quickly return the samples to a controlled environment, especially in field situations. In many cases, necessary manipulation of samples for analytical purposes can result in unwanted contamination, lost time and considerable costs associated with repeat experiments.

Conventional incubators are relatively large devices, having a size that is comparable to a small bar refrigerator. Typically, static cultures are formed on Petri dishes and then placed in the incubator on shelves. While the incubator may typically have temperature, humidity and gas control, there is generally no provision for optically inspecting the cultures without opening the incubator and removing the Petri dish from the incubator. This exposes the culture to possible contamination, and may cause other problems relating to culture growth. Hence, it would be desirable to have an improved centimeter-scale, diagnostics incubator that does not allow cross-contamination of the samples, and, has integrated perfusion capability to enable long-term culturing of otherwise difficult to culture biological samples such as tissue slices. It would also be desirable to have a centimeter-scale, integrated diagnostics incubator that permits in-situ optical inspection of cultures within the incubator and provides superior control of the cellular microenvironment during imaging.

Experiments involving physiologically faithful, thick, three-dimensional (3-D) in-vitro cultures are time constrained as the tissue decays metabolically in the absence of functional vasculature and perfusion, often well before the relevant studies have been completed. Thus, it would be desirable to a have a centimeter scale diagnostic incubator with integrated perfusion that prevents high-density cultures from decaying metabolically by actively controlling the nutrient medium exchange rate and enabling the forced convection, intercellular mass transport to overcome otherwise diffusion limited nutrient and gas delivery, and, temporally decreasing nutrient and gas concentration through the culture.

Since high-density cultures require many cells it would be desirable that the size of the cell culture chamber be as small as possible to reduce expenditures and enable high-plating densities with the low number of cells. It would be desirable that the centimeter-scale diagnostics incubator has small overall dimensions that would facilitate the control of the cellular microenvironment and provide the shortest possible response time to the physical changes and chemical stimuli. It would also be desirable to have a centimeter-scale, integrated diagnostics incubator that is portable and may be used in the field. Ultimately, it would also be desirable to have a centimeter-scale, integrated diagnostics incubator of a modular design to address different demands.

While 2-D culturing constitutes a standard practice for many fundamental studies, researchers are increasingly implementing the 3-D cell culture systems because they are biologically more realistic in capturing the in-vivo condition than their 2-D correlates. The three-dimensionality enables scientists to investigate cellular behavior in a more physiologically relevant state, while preserving the primary advantages of traditional in vitro systems, such as the control of the cellular environment, accessibility for imaging, and elimination of systemic effects. Cells cultured in a 3-D environment are found to better represent the in vivo cellular behavior than cells cultured in monolayers. This was shown for different kinds of cell lines, e.g. for fibroblasts cells by Grinnell F. in 2000 Trends Cell Biol 10:362-365; for breast cells, e.g., Wang F. et al. in 1998 Proc Natl Acad Sci USA 95:14821-14826; for osteoblastic cells, by Granet C. et al. in 1998 Med Biol Eng Comput 36:513-519; and, neural cells by Fawcett J. W. et al. in 1989 Dev Biol 135:449-458, or Fawcett J. W. et al. in 1995 Exp Brain Res 106:275-282.

The way cells interact with each other and their microenvironment is fundamentally different in 3-D and 2-D cultures, see for example Schmeichel K. L. et al. in 2003, J Cell Sci 11:2377-2388. In many cases these interactions are reduced or negligible in 2-D cultures. This necessitated the development of neural cell culture models to support high, 3-D cellular densities with cells evenly distributed throughout the full thickness of the matrix. The extracellular matrix material supports the cells in a 3-D setting, and, enhances cell-to-cell and cell-to-matrix interactions, e.g., O′Connor S. M. et al. in 2001 Neurosci Lett 304:189-193; Woerly S. et al. in 1996 Neurosci Lett 205:197-201. However, these cultures have relied on passive diffusion for nutrient delivery and removal of toxic waste products necessitating the use of cell densities much lower than those found in the brain for example. Therefore, diffusion limited mass transport in nutrient delivery prevented the development of more in vivo resembling 3-D neural cell culture models having high cellular density (≧10⁴ cells/mm³) and uniform cell distribution throughout culture thickness (>500 μm).

Growing demands for long-term incubation of physiologically faithful, three-dimensional (3-D) neuronal and other cultures during extended physiological studies require efficient perfusion platforms with functional vasculatures that mimic the in vivo condition in a thermally regulated environment. While expensive, relatively small, incubation baths with thermostatically controlled water jackets and capillary action perfusion are available commercially, to date, they remain incompatible with the microfabrication processes with their use confined to specific experimental conditions associated with the limits of capillary action perfusion. Representative incubation baths with passive perfusion are disclosed by Haas H. L. et al. in 1979 J Neurosci Meth 1:323-325, and, Zbicz K. L. et al. in 1985 J Neurophysiol 53:1038-1058.

The widespread use of 3-D neural cultures in medical research, however, is often hindered by low water solubility of oxygen, limited diffusion of media and oxygen through the tissue, and poor waste removal. Once the slices are being harvested, the utility of experiments is restricted by the quality of tissue perfusion, as the success of electrophysiological studies, for example, depends on long term slice viability to take reliable recordings.

There are generally two kinds of perfusion chambers that are being used to extend the viability of acute tissue slices in vitro based on a constant circulation of the culture medium by passively augmenting the supply of media and oxygen. In the first kind, the tissue is submersed in the bathing solution and perfused using oxygenated media as discussed by Richards C. D. et al. in 1977 Br J Pharmacol 59:526 P; Nicoll R. A. et al. in 1981 J Neurosci Meth 4:153-156; Palovcik R. A. et al. in 1986 J Neurosci Meth 17:129-139; Shi W. X. et al. in 1990 Neurosci Meth 35:235-240, for example. In the second kind, the tissue rests on a mesh at the interface between the open channel flow of perfusate, underneath the mesh holding the tissue, and, oxygenated and humidified atmosphere above the mesh. Representative chambers are described by Li C. L. et al. in 1957 J Physiol-London 139:178-190; Reynaud J. C. et al. in 1995 J Neurosci Meth. 58:203-208; Krimer L. S et al. in 1997 J Neurosci Meth 75:55-58.

Both approaches have practical benefits and shortcomings. The principal advantage of the submerged tissue incubation is faster diffusion of the bathing solution into the slice than in interface type chambers where only one side of the tissue is exposed to the media, not both. Although intuitively tissue would seem to have better oxygenation lying at the interface than being submerged, due to limited water solubility of oxygen in the latter, Croning M. D. R. et al. in 1998 J Neurosci Meth 81:103-11 found that the degree of disruption of ionic homeostatis by anoxia in rat hippocampal slices was greater when they were maintained at the interface. This could be attributed to the discontinuities in the flow of media associated with the surface tension effects at the gas-liquid interface. In addition to being simpler to design, submerged chambers provide more constant environment with less perturbations in fluid flow, purging of bubbles, and, draining, and, permit rapid exchanges of the bathing medium. Hence it appears that better perfusion fixation, in preventing both the starvation and anoxia during the process of observation, can be achieved in submerged chambers. Still, a major drawback in submerging the tissue below the liquid surface is actually keeping it submerged, because it will float unless it is properly attached to the perfusing substrate or restrained otherwise. In addition, localized injection of drugs is virtually impossible to administer with the current designs.

The dominant mode of mass transport in these scarcely used chambers remains diffusion and/or capillary action to passively augment the supply of media and oxygen to the tissue that eventually runs down metabolically. However, to meet and exceed the metabolic requirements for nutrients and the removal of catabolic waste products, nutrient delivery by pure diffusion becomes insufficient and demands a convective enhancement for an adequate, dynamic, long-term control of the culture condition. Whether or not forced convection nutrient injection increases the cellular adsorption is questionable, however, high exchange rates maintain sufficiently high intercellular concentration of nutrients throughout the culture thus preventing their metabolic rundown over the long-term.

### BRIEF DESCRIPTION OF THE DRAWINGS

The various features, functionalities and practical advantages of the present invention may be more readily understood with reference to the following detailed description taken in conjunction with the accompanying drawings, wherein like reference numerals designate like structural elements, and in which:

FIG. 1 is a top view of an exemplary centimeter-scale, diagnostics incubator with integrated perfusion;

FIGS. 1a and 1b illustrates various views of another exemplary incubator;

FIG. 2 is a top view of an exemplary microfluidic perfusion chamber that may be used in the diagnostics incubator and that comprises a culture chamber with fluidic ports and attached semi-permeable membrane that encapsulates the structure;

FIGS. 3 and 4 illustrate three-dimensional views of the exemplary perfusion chamber;

FIG. 5 is a cross-sectional view of the exemplary perfused culture chamber with integrated microfluidics;

FIG. 6 is a top view showing details of a cell culture life support chamber centrally integrated within the perfusion platform;

FIG. 7 illustrates an exemplary microscope stage insert plate that can be used to attach the centimeter scale incubator with an integrated perfusion chamber onto a microscope stage;

FIG. 8 illustrates an exemplary in-line auto-venting aerator and bubble trap that is used at high-exchange rates when apical gas exchange is not sufficient;

FIGS. 9a-l show particle image velocimetry measurements of the induced flow within the culture chamber at different elevations parallel to the perfusing substrate;

FIG. 10 shows graphs that illustrate thermocouple temperature measurements following an initial 10-minute long controller auto-tuning;

FIG. 11 shows graphs that illustrate temperature measurements while the incubator is in operation;

FIG. 12 illustrates a disposable thermopolymer master formed by solid object printing for use in elastomeric molding of perfusion chambers; and

FIG. 13 illustrates an exemplary in-line venting aerator bubble trap that may be employed with the incubator.

### DETAILED DESCRIPTION

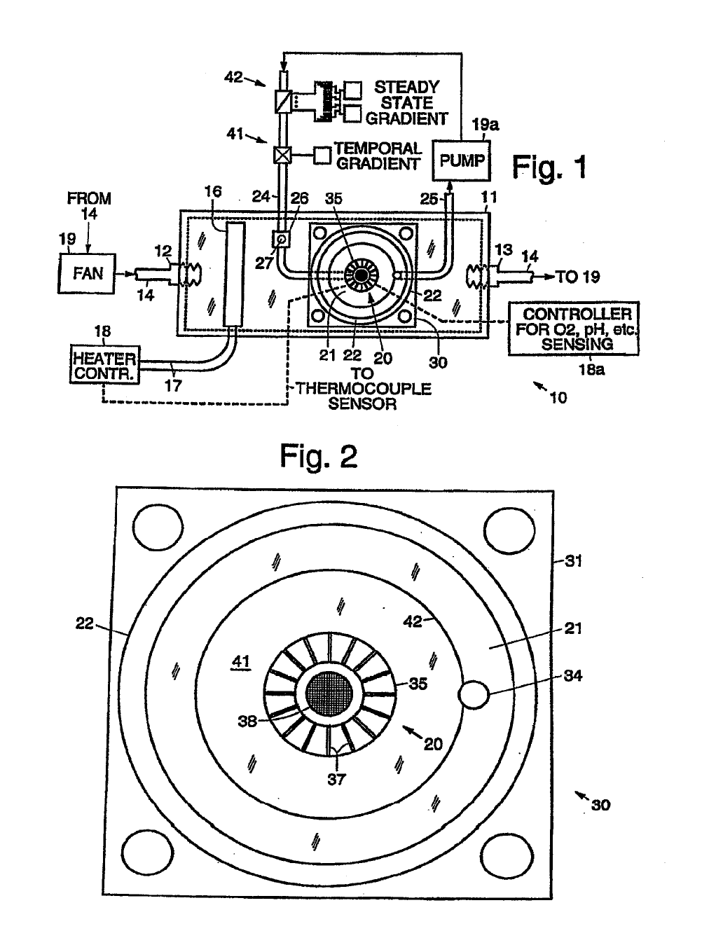

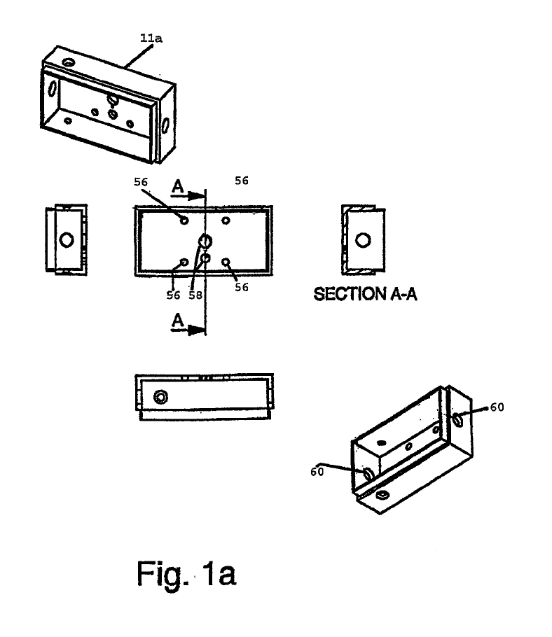

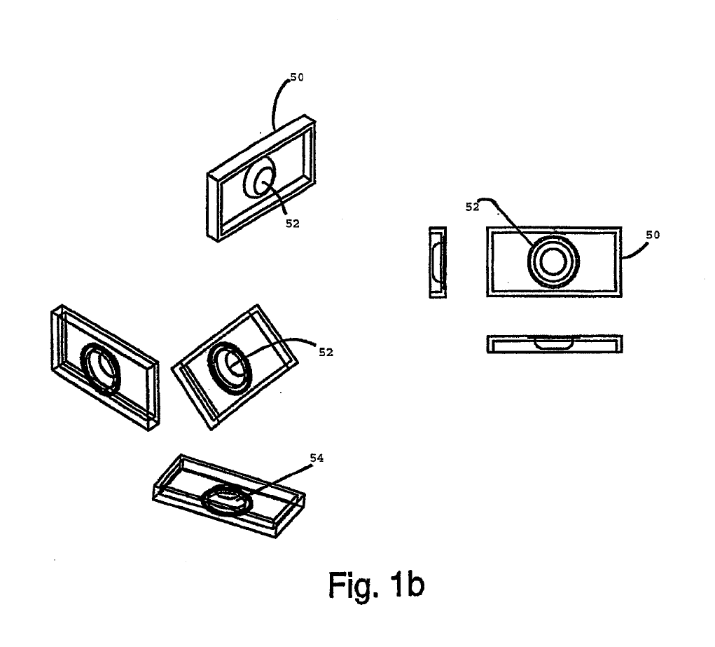

To overcome problems associated with conventional incubator technologies, and referring to the drawing figures, disclosed is a biocompatible, centimeter-scale, diagnostics incubator 10 having an integrated microfluidic perfusion chamber 30 which allows simultaneous culturing and experiments on a bench top or a microscope stand, with full control of the environmental conditions, fluidic, optical, and, electrical accessibility. By way of illustration, FIG. 1 shows an exemplary centimeter-scale, integrated diagnostics incubator 10 comprising an exemplary integrated perfusion chamber 30.

As is shown in FIG. 1, an exemplary reduced-to-practice centimeter-scale, integrated diagnostics incubator 10, or mini-incubator 10, comprises a biocompatible, polystyrene, heated enclosure 11 with exemplary outside dimensions of 5×2.5×1.8 cm (L×W×D). The enclosure 11 has a removable top cover that facilitates the integration and packaging. Owing to the clarity of the polystyrene material, all surfaces of the enclosure 11 are optically transparent and enable visual inspection.

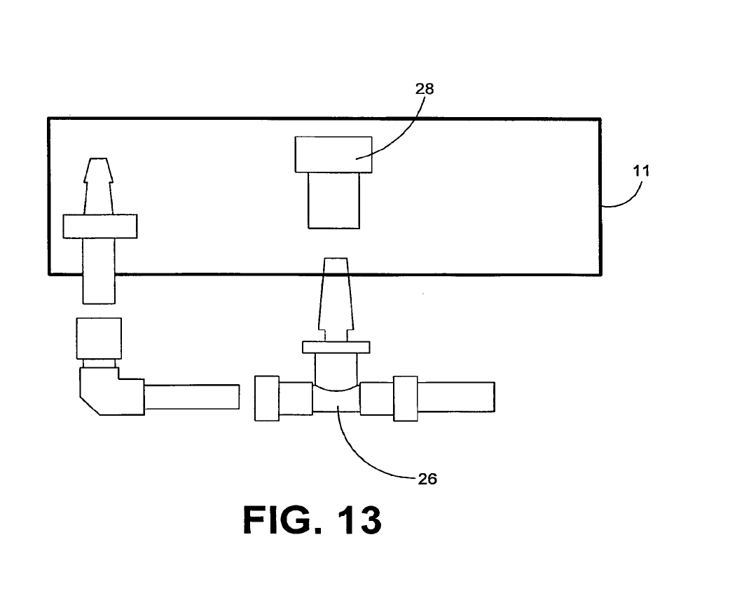

The incubator 10 houses a biocompatible polydimethylsiloxane (PDMS), for example, perfusion chamber 30 with a centrally placed cell culture life support perfusion chamber 20, a semi-permeable membrane aerator 21 that encapsulates the chamber 20, a miniature, thin flexible heater 16 that maintains a desired temperature within the incubator 10 or heated enclosure 11, thermocouple sensors connected to a temperature controller 18, and; pertinent gas 12, 13 and fluidic interfaces 24, 25. Incubator 10 may also have an integrated in-line venting bubble trap 26 (see also FIG. 13) to eliminate the non-dissolved bubbles from the media, and, a membrane aerator 27 to buffer and equilibrate the media with the gas environment inside the incubator 10 prior to nutrient medium injection into the perfusion chamber 30. The optically clear incubator enclosure 11 and a semi-permeable membrane aerator 21 permit optical examination or observation of a culture growing within the culture chamber 20. Imaging accessible, semi-permeable membrane 21 (aerator 21 or oxygenator 21), located on the top of the perfusion chamber 30, ensures sufficient gas delivery and provides sterile, moist microenvironment of appropriate acidity. By way of membrane aerators 21 and 27 both sides (top and bottom) of the cultured tissue are exposed to gases necessary for cellular metabolism.

On the sides of the heated enclosure 11 there are separate gas connectors that enable the appropriate gas mixture to enter and leave the incubator 10 at a desired volume flow rate. A forced convection flow of gases, such as 5% CO₂-air mixture for example, enters through gas inlet 12 and outlet port 13. The gas inlet 12 and outlet 13 ports are coupled to gas lines 14 that lead to a separate miniature box (not shown) that houses a fan 19 and contains prescribed gas mixture via shut-off valve connection to a gas tank. A fan 19 can be a pair of miniature blowers with the overall dimensions of 4 cm×4 cm×4 cm used in electronics cooling (Thermaltake A1899, for example). Blowers force the appropriate gas mixture through the gas lines 14 and optically clear enclosure 11 and allow external control of the fan speed to provide an optimal gas rate at a minimal noise level.

There are two fluidic interfaces for the nutrient medium, for example. They are associated with continuous infusion of the nutrients into the perfusion chamber 30 and withdrawal of used media and metabolic waste products at the same rate. All fluidic components (perfusion chamber 30, aerators, fittings and tubes) are steam autoclavable (re-usable) with stiff microbore tubes fabricated in an inert (Teflon FEP) material that minimizes the problems associated with bio-fouling (non-specific protein adhesion along the inner walls). Fluidic inlet and outlet ports 24, 25 are coupled to the infusion and withdrawal side of the syringe pump moving block respectively. An exemplary syringe pump 19a with opposing syringes on a single drive may be a KdScientific pump model KDS210, for example. The pump 19a mode of operation pertinent to the particular cell feeding schedule is externally controlled and can be a continuous infusion and withdrawal; programmable, time-periodic or aperiodic infusion and withdrawal; or, push/pull; with or without the re-use of spent perfusate. In either mode, while one syringe infuses the media, the other syringe withdraws the perfusate at the same rate so that the pressure within the cell culture chamber 20 remains at or near the atmospheric level. This particular mode protects the cultures from prohibitively high pressures otherwise associated with the return flow from the chamber. In a portable design, syringe pump 19a can be replaced with miniature piezoelectric pumps that can be battery operated (e.g., Bartels Mikrotechnik GmbH micropump measures only 14×14×3.5 mm and provides flow rates of 50 μl/min to 3 ml/min using 3V DC battery at 30 mA) and fluid delivery rates manipulated by varying the amplitude or the frequency, or both, of the power supplied using an inexpensive custom built function generator based on the operational amplifier circuits, for example.

Unlike commercially available (Zbicz- or Haas-type) perfusion chambers, disclosed manufacturable, cost efficient, disposable or autoclavable, microvascular perfusion chambers 30 can be easily configured to meet specific demands while exceeding the available exchange rates of commercial devices by a few orders of magnitude. Fluidic functionality of the perfusion chamber 30 enables the culturing of thicker tissue slices and engineered 3-D culture models that better represent the in-vivo condition via forced convection intercellular mass transport that is not feasible in commercially available interface or submerged-type perfusion chambers. A simple fabrication methodology based on elastomeric polymer molding allows the integration of microfluidic and MEMS components (not shown) into the perfusion chamber 30, such as micropumps, valves, mixers, microinjectors, cellular manipulators or multielectrode arrays, for example. It also facilitates the interfacing of perfusion chambers 30 and incubators 10 and enables the integration of a number of plug-in functionalities into the mini-incubator 10 tailored to specific use in a short turn-around time.

The disclosed fluidic design also enables the introduction of externally controlled temporally varying 41, or, spatially varying, temporally invariant concentration gradients 42 of injected agents into the culture chamber 20, with fluidic infrastructure and control located outside of the incubator enclosure 11. Owing to the flexibility of the perfusion system, the nature of cell feedings can be radically changed. For example, a programmable time-periodic injection of nutrients can be employed, followed by times where perfusion chamber 30 acts as a static control; the media could also be cycled in and out of the chamber 30 in some periodic or aperiodic fashion to break the temporal invariance of spatial gradients; or, the ratio of fresh to used media can be varied to increase the amount of cytokines (neurotrophins, for example) if the need arises. The fluidic system may be easily adapted to address and individually control multiple culture chambers 20 or perfusion chambers 30.

Drugs can be discretely applied to specific regions using separately addressable fluidic inputs, interfaced outside the incubator 10 and introduced into the perfusion chamber 30 through the inlet port 33 or a plurality of inlet ports 33 (not shown), as could other substances. This makes the centimeter-scale incubator 10 ideal for time-dependent pharmacological studies at dynamically controlled exchange rates directly on a microscope stand, for example. Extra- and intercellular ion concentration can be accurately controlled and easily altered. By collecting the spent perfusate, substances released during stimulation can be easily analyzed. Collected perfusate can be mixed with the fresh medium and re-used, particularly at high exchange rates and cell densities when neurotrophins may be depleting.

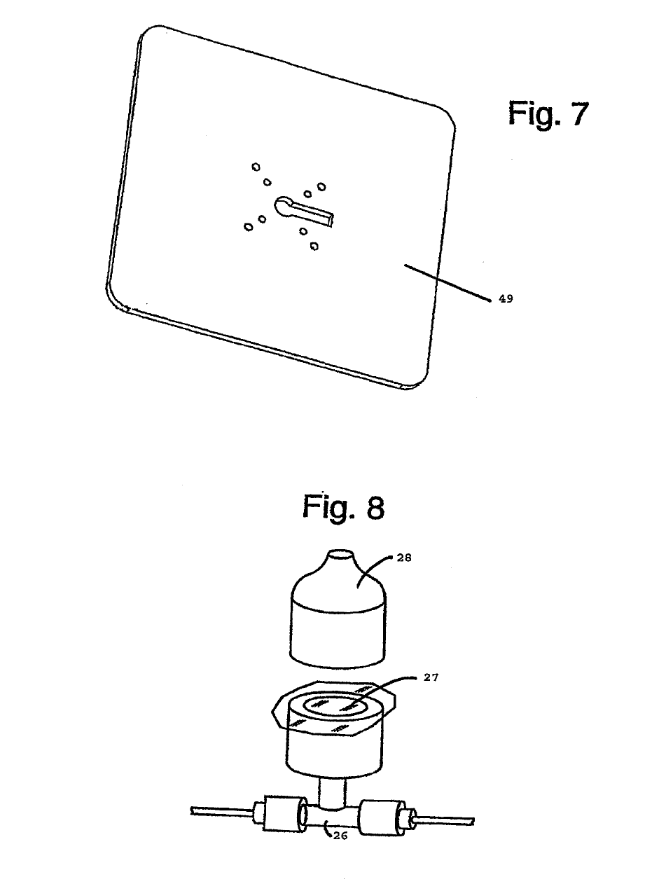

The centimeter-scale incubator 10 with its integrated perfusion chamber 30 permits simultaneous studies of cell culture parameters using optical diagnostics. For this purpose, as depicted in FIG. 7, a particular microscope stage insert 49 may be used. The incubator 10 or perfusion chamber 30 connects directly to the microscope stage insert plate by way of 4 screws and nuts that pass through four through-holes located on the body 31 of the perfusion chamber 30 and centimeter-scale incubator 10. This is shown in FIGS. 1a and 1b which illustrate various views of another exemplary incubator. This enables in situ cell culture viability studies and immunocytochemistry, for example, below the microscope objective. The exemplary microscope stage insert 49, shown in FIG. 7, was built using stereolithography.

As shown in FIG. 1a, shown is a bottom portion 11a of the incubator enclosure 11 (FIG. 1) that includes holes 56 that facilitate mounting of the perfusion chamber 30 (FIG. 2). Also, holes 58 accommodate thermocouples, a media inlet tube, a media outlet tube, or other structures. Also, the bottom portion 11a includes tapped holes 60 to receive gas fittings to circulate gasses through the enclosure 11.

Although clear incubator enclosure 11 enables optical diagnostics on a microscope stand, and, the visual inspection of the cultures growing inside the incubator 10, high-resolution optical imaging demands different configuration that allows the microscope objective to come in direct contact with the semi-permeable membrane 21 that encapsulates the perfusion chamber 30. To investigate biochemicals down to molecular level or growing cultures on a single cell basis, high numerical aperture objectives are routinely used. These oil or water immersion objectives have very short working distances that are at the order of a couple of millimeters at most. As this falls within the thickness of the incubator enclosure cover top, cultures cannot be imaged.

Referring to FIG. 1b, to overcome this issue the top cover 50 of the incubator can be modified to include a circular through-hole 52. The through hole 52 can be covered with a gas, water and oil impermeable membrane 54. Optically clear, impermeable membrane is sealed around the perimeter of the through-hole formed in the top cover of the incubator enclosure using an O-ring. Impermeable membrane is stretched loosely over the through-hole during incubation. During high-resolution imaging, one side of the impermeable membrane (not facing the culture) is covered with oil or water depending on the type of the objective used. Immersed, high numerical aperture objective is then placed in contact with the impermeable membrane. The opposite side of the impermeable membrane then comes in direct contact with, and, becomes stretched over the membrane aerator 21. The membrane aerator 21, U.S. Pat. No. 6,521,451 (12.7 μm thick Dupont Teflon® FEP fluorocarbon film) is fairly impermeable to water (0.004% water absorption, using ASTM D570 test method) and water vapor (permeability of 7 g/m²/day, ASTM E-96) and quite permeable to oxygen, nitrogen and carbon dioxide (respective permeabilities of 298, 125 and 647.5 cc-mm/m²-24 hr-atm using ASTM D-1434 test method). Stated values are for 25 μm thick film. With the visible transmission of 96% (ASTM E-424) this selectively permeable membrane is ideal for optical imaging and culture aeration. The impermeable membrane, such as 12.7 μm thick Saran Wrap™ 3 Plastic Film (Dow Plastics) has a high transmission in the visible range (86% based on ASTM D1746) and is quite impermeable to gases and water vapor. Respective permeabilities of oxygen (ASTM D3985), nitrogen (ASTM D1434) and carbon dioxide (ASTM D1434) are 0.472, 0.0709 and 2.13 cc-mm/m²-24 hr-atm. Water vapor permeability is limited to 8.7 g/m²/day, Permatran W). Hence, this relatively impermeable thin film that replaces a part of the incubator enclosure 11, prevents gas exchange between the ambient and the incubated (heated) environment. SARAN WRAP™ 3 Plastic Film is a clear biaxially oriented monolayer barrier film, designed for tight wrap and heat-sealing applications with negligible air transmission of 0.14 cc-mm/m²-24 hr-atm. When imaging is not under way, the impermeable membrane remains well above the membrane aerator 21 and the efficiency of gas exchange remains unaltered compared to polystyrene top cover. During high-resolution imaging, direct contact between impermeable membrane and membrane aerator 21 may reduce sample exposure to gases depending on the relative surface area of the objective lens and perfusion chamber. For most of the objectives, however, the membrane aerator 21 top surface is not fully covered and much of the gas exchange now takes places laterally via microchannels 37 formed in the body of perfusion chamber 30. When impermeable membrane is being used for high-resolution optical analyses it is often desirable that the incubator enclosure 11 be opaque to prevent ambient light to reach the specimen and create background noise. Thus, clear polystyrene incubator enclosure 11 can be replaced with an opaque enclosure during life-time fluorescence imaging for example. A heating element 16, such as a miniature (2 cm×2.5 cm) resistive foil heater 16 (Minco HK5291, for example) is disposed within the enclosure 11 and has the lead wires 17 extending outside of the enclosure 11 to a temperature controller 18 coupled to a DC switching relay and a DC power supply that can be replaced with a battery for portability. Typical power requirements for the heating element 16 integrated within the incubator 10 are at the order of just 1 W.

To enable sample preservation, the resistive heating element 16, can be replaced with a thermoelectric pump such as Ferrotec Corporation miniature Peltier cooler (part #9502/065/012 M measuring 12×11×3 mm with cooling/heating capability of about 6 W at 1.2 Amps and the maximum temperature difference of 70° C.) to maintain the temperature below the ambient. Thermoelectric heat pumps can be used to either cool or heat the sample depending on the preservation/incubation requirements by simply changing the polarity of the voltage source. Slightly larger thermoelectric modules obtain temperature difference of 100° C. or more with heating/cooling capacity of few tens of Watts. Thermoelectric pumps can be battery operated for portability.

Although much of the text disclosing the incubator 10 focuses on 3-D culturing in perfused culture chambers 20, the utility of the incubator 10 is much broader and extends well beyond the realm of tissue engineering. For example, in some applications fluidic functionality may not be necessary and perfusion chamber 30 can be replaced with a PDMS specimen holder similar to a cylindrical culture well, and, encapsulated by a membrane aerator 21. By doing so various chemical, biochemical or even biological specimen can be preserved at specific atmosphere of gases at controlled temperature, but, without perfusion functionality. The use of thermoelectric pumps, for example, extends the use of the miniature incubator 10 to preservation and observation of viral or bacterial samples collected in the field prior to their inspection in full-scale laboratories. Although not shown, gas interfaces 12 and 13 can be connected to miniature quick-disconnect shut-off valve couplings (such as those from Kent Systems) attached to sterile filters, to prevent the infection from spreading out during common handling, or, gas bottle replacement in the field when the specimen requires long-term preservation. This also facilitates the interfacing of gas lines 14 with the miniature incubator 10 with a low-risk of contamination. In an even more robust approach to take bacterial or viral samples in the field, the perfusion chamber 30 can be replaced with a miniature pipette whose tip protrudes through the incubator enclosure, with acquired sample delivered inside the incubator 10 and sealed from the ambient using sterile shut-off couplings.

When cellular perfusion is not necessary, cylindrical well that replaces the perfusion chamber 30, can be coated with chemoattractants or chemorepellants and easily turned into a haptotaxis chamber to investigate the directional motility or outgrowth of cells cultured in monolayers. These gradients are established by altering the concentration of cytophilic adhesion sites on a polymer substrate. For example, in case of axonal outgrowth, either up or down a gradient of a substrate-bound chemoattractant.

For pharmacological studies, the perfusion chamber 30 may be replaced with a miniature chamber for steady-state gradient generation (see U.S. Pat. No. 6,705,357, for example). With minor modifications this utility reveals at what concentration drugs become effective, and, facilitates the maintenance of samples, repeatability of experiments, and, enables optical analyses of the cultures or other specimen over extended periods of time.

The temporal gradient generating device 41, or steady-state gradient generating apparatus 42 can be placed within the incubator 10 to replace the perfusion chamber 30. These devices can be easily turned into chemotaxis chambers to investigate the movement of cells, bacteria, and other single-cell or multicellular organisms towards certain intermittently or continuously injected chemicals into their microenvironment. Specific concentration of chemicals in question can be easily determined in apparatus 41 or 42 by optically inspecting the specimen.

In another embodiment, Brewer G. J. et al. in 2006 at the 2006 Annual Fall Meeting of the Biomedical Engineering Society and Rowe L. et al. in 2007 Lab Chip 7: 475-482; Rajaraman S. et al. in 2007 J Micromech Microeng 17:163-17, perfusion chamber 30 can be modified and used for packaging of different perfusion substrates such as miniature tower arrays having fluidic and electrical functionality, to feed and record from the cultures.

In applications where temperature sensitive diffusion studies are under way and small temperature gradients across the sample cannot be tolerated, the internally placed heating element 16, can be replaced with optically clear heaters based on Indium Tin Oxide (ITO) technology. ITO heaters, such as Honeywell 78000 Series or Dontech ThermaKlear™ Transparent Heaters, can be bonded to all interior surfaces of the incubator enclosure 11. By doing so the active volume of the incubator enclosure 11 can be reduced by a half or more. Volume reduction further facilitates the temperature control and the maintenance of isothermal condition within the incubator 10. Owing to their clarity, ITO heaters attached to the walls of the incubator enclosure 11, do not compromise optical diagnostics on a microscope stand or visual inspection of the samples. However, the material of the incubator enclosure 11 needs to be replaced with that of polycarbonate or some higher heat-resistant plastic. This could potentially render the entire cm-scale incubator apparatus 10 fully autoclavable.

For high-throughput processing of a large number of perfused samples with a low-risk of cross-contamination at isothermal conditions, the incubator enclosure 11 may have double walls. A number of perfusion chambers 30 with their respective membrane aerators 21 can be placed within inner enclosure. Inner enclosure is connected to a bottle having prescribed concentration of gases. ITO heaters can be attached to all interior surfaces of the outer enclosure or a plurality of thermofoil heaters can be placed in the clearance between the inner and the outer enclosure. A miniature blower can be attached to one side of the outer enclosure to recirculate air flowing between the inner and the outer enclosure, thus enabling forced convection over the heaters. In this case an external box that houses the mini-blowers is not necessary. Each sample may be perfused separately using miniature pumps to prevent the infection.

To render the diagnostic incubator 10 with integrated perfusion fully portable to take biological samples in the field, pertinent hardware in its simples form entails three components: a pump, a gas bottle and a heater controller along with their batteries.

Although not shown, additional sensors and controllers including but not limited to glucose consumption, pH or dissolved O₂ sensing and control 18a can be easily integrated into the perfusion chamber 30 and the enclosure 11 depending on particular demand. The modular design allows integration of all or some of the above mentioned elements and enables the packaging of a larger number of perfusion chambers 30 into a common, environmentally regulated enclosure 11. Higher number of independently controlled perfusion chambers 30, within an enclosure 11 enables high throughput processing of a number of cultures onboard a single, disposable platform.

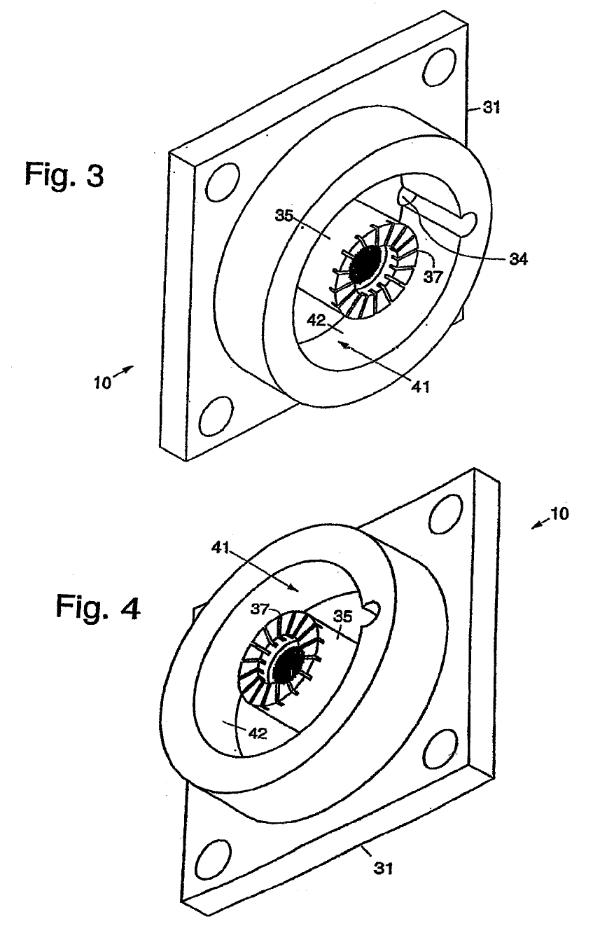

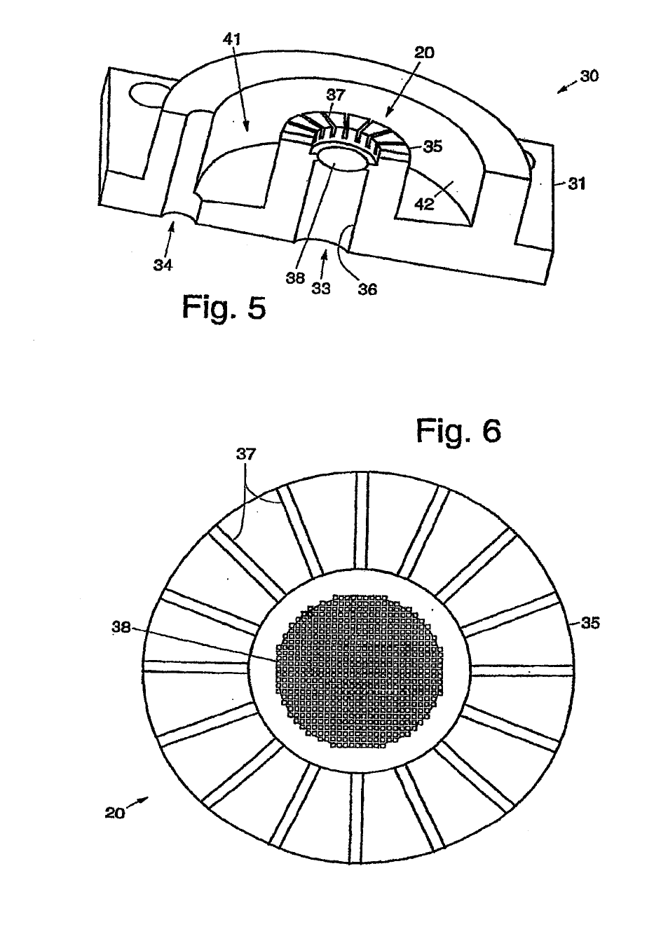

FIG. 2 shows top view of the exemplary microfluidic perfusion chamber 30 and a cell culture life support chamber 20. FIGS. 3 and 4 illustrate three-dimensional views of the perfusion chamber 30. FIG. 5 is a cross-sectional view of the perfusion chamber 30 and a cell culture chamber 20. FIG. 6 illustrates a top view showing details of the culture chamber 20 centrally formed in the perfusion chamber 30.

As is shown in FIG. 2, the exemplary perfusion chamber 30 comprises a body 31, which may be made of polydimethylsiloxane (PDMS), for example, and a semi-permeable membrane holder 22 that encapsulates the perfusion chamber 30. A central opening is formed in the body 31 that defines the inlet port 33 (FIG. 5) with the exit port 34 (FIG. 5) embedded into the bottom of the return flow (consisting of the used media and catabolites) collecting pool 41. The return flow collecting pool 41 is formed in the body 31 between cylindrical enclosures 35, 42. The fluidic inlet and outlet ports 33, 34 are coupled to a syringe pump 19 via inlet and outlet tubing 24, 25 (shown in FIG. 1). An optically clear membrane 21, such as a hydrophobic, fluorinated-polyethylene-propylene (FEP) membrane 21, see for example, Potter S. M. et al. in 2001 J Neurosci Meth 110:17-24, is attached to the top of the body 31 via miniature Teflon holder 22 with O-rings to seal the chamber 30 (see FIG. 2). The transparent membrane 21 contacts the top surfaces of the perfusion chamber 30 and culture chamber 20 while O-rings seal the device exterior wall 42 of the body 31, and thus the entire perfusion chamber 30. The FEP membrane 21 is impermeable to the media but permeable to gases necessary for cellular metabolism. The transparent membrane 21 secured with a Teflon holder 22 permits optical examination or observation of the tissue within the culture chamber 20. Such observation is not possible using conventional, large-scale incubators. Typically, culturing takes place in the incubator and tissue has to be taken from the refrigerator size incubator to be observed and analyzed under a microscope.

The body 31, as shown in FIGS. 3 and 4, of the perfusion chamber 30 is configured to have a centrally located cylindrical, cell culture life support chamber 20, an outer inclined, collecting pool 41, and, fluidic inlet and outlet ports 33, 34 shown in FIG. 5. The culture chamber 20 as schematically shown in FIG. 6 is an actively controlled incubation bath bounded by the perfusing substrate 38 that supports the tissue, located at the bottom of the culture chamber 20, cylindrical sidewall 35 that confine the culture to the area immediately above the substrate 38, and the optically clear semi-permeable membrane 21 attached to the top of the perfusion chamber 30 and held by the Teflon holder 22. A smooth, continuous, circulation of the media and analytes is achieved by using a simultaneous push-pull pumping system in which the output exactly balances the input, that is, the culture feedings are in mass equilibrium with the amount of retrieved perfusate. The level of bathing medium within the culture chamber 20 is controlled by the suction side of a syringe pump 19a. Nutrients enter the perfusion chamber 30 through the inlet port 33 before being distributed through the perfusion substrate 38, and further, throughout the volume of the culture chamber 20 (see FIG. 5). Used media and metabolic waste products are withdrawn from the cultured chamber 20 through an array of microchannels 37 that are incorporated into the walls of the cylindrical PDMS enclosure 35 bounding the culture chamber 20. Retrieved perfusate is collected within the inclined pool 41, outside the culture chamber 20, before it is withdrawn through the outlet port 34. The bottom surface of the exit flow collecting pool 41 is slanted to allow the used perfusate accumulating in the pool 41 to migrate toward the outlet port 34 formed in an exterior wall 42 of the body 31. A 6° inclination, for example, of the bottom surface of the pool 41 helps in steering the used media and catabolites towards the outlet port 34.

The cylindrical sidewall 35 that surrounds the culture chamber 20 measures 3.5 mm in diameter and raises 700 μm above the perfusion substrate 38 so that the exemplary, effective volume for 3-D culturing amounts to about 7 μl. A tissue slice or a tissue-equivalent constructs is adhered to the interior of the culture chamber, i.e. see Cullen D. K at al. in 2007 J Neural Eng 4: 159-172. Media enters the culture chamber 20 from an array of micronozzles, normal to the perfusing substrate 38 (FIG. 6) that upholds the tissue, and exits through plurality or radial slots 37 or microchannels 37 formed within the cylindrical enclosure 35 that confines the tissue laterally. Exemplary 150 μm wide microchannels 37 are 375 μm deep and start at the elevation of 375 μm above the perfusion substrate 38. These microchannels 38 allow used cell culture media to leave the culture chamber 20. The start of the microchannels 38 at a mid-height of the cylindrical enclosure 35 enables the tissue to always be at least partly submerged. By filling the entire culture chamber 20 first, without starting the suction, one can ensure that the culture is fully submersed prior to the start of a closed-loop circulation. The radial exit flow through the microchannel array 37 peripherally located within the culturing chamber 20 is facilitated by the presence of a selectively permeable membrane 21 placed over the top of the perfusion chamber 30. While traditional perfusion platforms rely mainly on diffusion and/or capillary action, the incubator 10 disclosed herein provides an efficient control of both convective and diffusive mass transport relying on interstitial nutrient convection induced by an array of microjets issuing from micronozzles formed in the perfusing substrate 38 that upholds the tissue and peripheral extraction of the perfusate, by an array of microchannels 37, for example, located within the cylindrical enclosure 35 that laterally confines the tissue. Actively growing high density cultures are continually perfused with the fresh medium. Volumetric flow rates for neuronal-only and neuronal/astrocytic co-cultures are about 3 exchanges per hour or more depending on the type of the culture incubated and cellular density. The cell longevity is the dominant factor determining the flow rate of the supplied media. Collected perfusate can be mixed with the fresh medium and reused, particularly at high exchange rates and cell densities when neurotrophins may be depleting.

The perfusing substrate 38 may be made of gold, for example, and bonded to or otherwise sealed to the lip formed at the bottom of the culture chamber 20. An exemplary gold perfusion substrate 38 (such as PELCO® center-marked grids, 300 mesh, 1GG300, for example) measures 3 mm in diameter and contains 54 μm square openings (micronozzles) with center-to-center spacing of 85 μm. This yields 40% open area for fluid flow. Different perfusion substrates 38, of different topology and surface chemistry for example, may be used to promote cellular adhesion to the bottom of the culture chamber 20 and enable perfusion of the tissue cultured inside. Micronozzles can have sub-cellular dimensions, i.e., 5 μm for example, which is well below the size of the neural cell body (about 10 μm). Various kinds of commercially available or custom made substrates 38 having different porosity and hydraulic diameters, can be bonded to the bottom of the culture chamber 20 using a thin layer of PDMS pre-polymer/catalyst mixture. This allows manipulation of the cellular microenvironment by altering the flow conditions and cellular adhesion by way of substrate-bound cytophilic coatings. A variety of biocompatible materials, other than gold, may be used as perfusion substrates 38 and to support the cells cultured within the culture chamber 20. Thin porous films made of nylon, polycarbonate or polytetrafluoroethylene (PTFE) constitute one of many possible approaches. In some cases selective coating of these substrates 38 promotes preferential cell growth if desired. Cell attachment may be enhanced using a variety of biocompatible coatings, depending on the exact nature of the study and the type of cells cultured within the culture chamber 20. Dissociated cells can be grown in synthetic, biodegradable extracellular matrices (ECMs) placed above the perfusing substrates since most of the ECM materials adhere well, see for example, O′Connor S. M., et al. in 2001 Neurosci Lett 304:189-193; or, Woerly S., et al. in 1996 Neurosci Lett 205:197-201. Experimental systems that reconstitute 3-D cell-cell interactions and control tissue formation in vitro and in vivo are critical to tissue engineering applications. Synthetic ECMs in the form of 3-D scaffolds (matrigel), hydrogels (such as agarose), and, fiber-like biodegradable polymers such as polylacticcoglycolic acid (PLGA) or polylactic acid (PLLA) can be used to culture high-density 3-D dissociated cultures over the perfusion substrates and their ability to regulate tissue formation can be easily observed. With perfusion, 3-D cultures composed of cells of interest, matrix secreted by these cells, and biodegradable, synthetic ECM can be used to study the role of intercellular signaling in tissue development and may be used in clinical applications as replacement to lost or injured tissue.

The perfusion chamber 30 is aerated by a hydrophobic, fluorinated-polyethylene-propylene (FEP) membrane 21 (see U.S. Pat. No. 6,521,451, for example) that is selectively permeable to gases such as oxygen, nitrogen, hydrogen and carbon dioxide and relatively impermeable to microbes, water and water vapor (less than 0.01% moisture absorption). Multiple functionalities of the FEP membrane 21 eliminate the need to equilibrate the media in an external bath, the most frequently used type of aerator (oxygenator for acute slices) used in conventional perfusion chambers, incorporate bubble traps, or to include the gas lids to direct the moist and saturated air over the culture like in interface-type (Haas) chambers. Separate buffering of the media to achieve the appropriate acidity is also deemed unnecessary. Hence, the use of this simple, yet efficient, membrane aerator 21 enables a fine control of the cellular climate without complicated, bulky components, thus allowing the fabrication of inexpensive compact devices. Other benefits of an aerator comprising an FEP membrane 21 and a membrane holder 22 include the ability to culture in a non-humidified incubator since the media does not evaporate from the culture chamber 20 thereby reducing the risks associated with the contamination in a humid environment, and, preventing the increase in osmotic strength that can be detrimental to the cultures. Optically transparent FEP membranes are also impermeable to microbes resulting in a reduction of contamination occurrences making them ideal for microscopic imaging in a sterile environment. Different membrane materials and/or thicknesses may also be used as efficient, optically clear, aerators/bubble traps.

In all microfluidic devices the purging of bubbles becomes a difficult problem, and while commercially available perfusion chambers usually incorporate external bubble traps, the FEP membrane 21 used in the culture chamber 20 enables sterile venting of small volumes, i.e., it constitutes an efficient auto-venting bubble trap on its own. To further reduce the amount of non-dissolved gas bubbles entering the culture chamber 20 and equilibrate the fresh media before being injected into the perfusion chamber 30, an in-line bubbler/outgassing device can be inserted into the perfusion circuit. For example, the male luer slip side of the barbed connector (Qosina® 17611 polypropylene) inserted into the inlet of the perfusion chamber 30, connects to a female luer end of an L luer adapter (Qosina® 17610 polycarbonate). Horizontal leg (male slip) of this L adapter connects to a female side of a luer tee 26 having two male slips (Qosina® 88216 polycarbonate). Horizontal male slip of the luer tee 26 is connected to the infusing syringe via microbore tubing and luer tight female fittings (Upchurch Scientific' P-835). The vertical leg of the medical grade luer tee 26 is used to form an in-line vented aerator/bubbler/outgassing device. To assemble the device an FEP membrane 27 is stretched over the vertical leg of the tee 26 having a male slip luer end, and, sealed with a finger-snap vented female luer end-cap 28 (Qosina® 71681 polypropylene).

This device prevents the bubbles from reaching the culture chamber 20, and ensures the media equilibrium with the gas environment inside the cm-scale incubator 10. To ensure that the media is buffered and equilibrated before entering the culture chamber 20 at high exchange rates, a larger surface area for the in-line gas exchange can be obtained alternatively. For example, an off-the-shelf filter holder (such as 13 mm Swinnex filter holder, Millipore, Billerica, Mass.) may sandwich a 12.7 μm thick hydrophobic film (Teflon® FEP film, Dupont, Circleville, Ohio) and seal it with an O-ring between the holder top- and bottom piece. This filter holder with a sandwiched FEP membrane 27, as is illustrated in FIG. 8, has a male luer-like slip that is received by a tee having a vertical female luer leg (e.g. Qosina® 80061 polycarbonate tee). When the inlet to the filter is kept in the upright position, the membrane aerator 27 helps the venting of perfusing lines thus eliminating the need for a bubble trap. This also enables a smooth circulation of the bathing medium using a syringe-rather than a peristaltic pump, as peristaltic pumps generally induce more fluctuations in nutrient delivery.

The presence of the FEP membrane 21 on the top of the culture ensures that the plethora of gas is provided from the top as is from the bottom via perfusing medium. In addition, the PDMS material used in fabricating the chamber 30 is found to be highly permeable to oxygen and carbon dioxide so that the cellular exposure to the gases is, in fact, enhanced from all exposed sides of the tissue. Hence, unlike in interface-type chambers where sufficient gas exchange is hindered by surface tension induced flow perturbations, the perfusion chamber 30 exposes both sides of the tissue to relevant gases while keeping the tissue submerged. Compared to Zbicz-type chambers where submerged tissue may suffer from anoxia due to limited water solubility of oxygen for example, in culture chamber 20 one side of the tissue is fully exposed to the gases above the FEP membrane 21, with the other side convectively bathed in equilibrated nutrient medium at high exchange rates. This enhances the thickness of slices that can be investigated in vitro compared to conventional chambers, and, enables optical studies and tissue imaging.

All components of the PDMS perfusion chamber 30 and those of relevant fluidics hardware are reusable and steam autoclavable, although the low cost of fabrication allows it to be a disposable device. The inlet and outlet fluidic connections are removable, i.e. they can be easily pushed in or disconnected from the perfusion chamber 30, which facilitates the rinsing of tubes and the perfusion chamber 30 prior to steam autoclaving them. The inlet and outlet ports 33, 34 are designed to accept standard 3/32″ and 1/16″ barbed, nylon adapters with 10-32 male threaded, receiving ports, respectively. These adapters can be either straight-through, for vertical tube connection, or elbow-like, to connect the tubes at a side. The adapter type depends on the spatial constraints during imaging on either upright or inverted microscopes with episcopic illumination source, such as epifluorescence. An insert plate can be custom made to match a particular microscope stage, see exemplary insert plate in FIG. 7, thus allowing perfusion experiments, injection of drugs, stimuli, cytokines and other reagents to be introduced during imaging. A receiving tefzel, 10-32 female-to-luer adapters connect to polypropylene LuerTight fittings (Upchurch Scientific). These quick-disconnect fittings conform to medical luer taper configurations with simple connection to luer lock syringes. Exemplary fittings accept 1/16″ OD semi-rigid fluoropolymer tubing such as the inert, Teflon FEP, transparent tubes with 100 to 500 μm microbores. If soft tubing is to be used, then the fittings are less bulky with barbed chamber-to-tube connectors, and, luer-to-barb adapters to connect tubes to syringes. Barbed fittings require larger bore tubes, thus increasing the amount of dead volume in the system, and when the amounts of used reagents become important, the use of harder tubes is definitely warranted. While the interfacing of the perfusion chamber 30 with the cm-scale integrated, diagnostics incubator 10 is fairly straightforward, hard tubing facilitates the placement of the incubator 10 onto a microscope stage with no interruptions to the flow whatsoever.

### Velocity Measurements

The perfusion chamber 30 and incubator 10 exposes the cells cultured in a culture chamber 20 to a continuous, direct flow of media through an array of micronozzles formed by the orifice plate 38 and creates 3-D, convectively biased circulation towards the perimeter of the culture chamber 20 where perfusate leaves through an array of microchannels 37. The flow under consideration is studied experimentally using microscopic particle image velocimetry, μ-PIV (see for example, Wereley S. T. et al., Micron resolution particle image velocimetry, in Diagnostic techniques in microfluidics, Editor K. Breuer, New York, Springer Verlag, 2004) to obtain reliable and reproducible two-dimensional velocity fields in the planes normal to the optical axis with high accuracy and relatively high spatial resolution. PIV is a well accepted, non-intrusive measurement technique where the fluid velocity is measured by recording the displacement of small tracer particles added to the fluid under the assumption that the particle density is identical or commensurate to that of the surrounding fluid, and its size small enough to follow the flow faithfully without influencing the flow itself (low Stokes number). Microscopic PIV is a modification of a standard PIV technique to enable spatially resolved measurements of instantaneous velocity distributions in sub-millimeter scale flow domains and allow detection of micron-scale spatial structures within the flow.

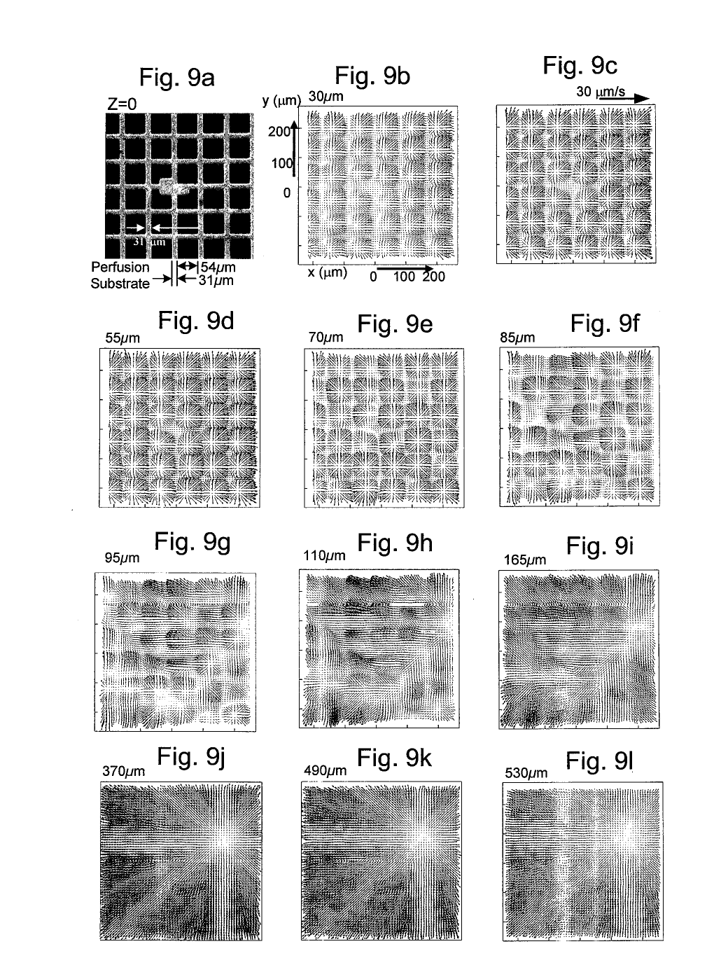

In the present experiments velocity resolution is within 5% of the microjet ejection velocity. Velocity distributions in the x-y planes, parallel to the perufsing substrate 38, and normal to the axes of microjets emanating from about 570 square micronozzles formed at the perfusing substrate 38, are shown in FIGS. 9a-1 with the elevation, z, measured from perfusing substrate. The field of view measures 450×457 μm and covers the central part of the pefusing substrate 38, FIG. 9a, with an array of about 6×6 nozzles in focus. The nominal volume flow rate through the perfusion chamber is 5 μl/min with micro-jet ejection velocity of 30 μm/s and a nozzle based Reynolds number of 0.002. A small culturing volume (about 7 μl) allows rapid exchange of perfusate (about 40 exchanges per hour for the present experiments) facilitates control, and reduces the amount of spent media.

The dynamics of induced flow within the culture chamber 20 varies with elevation. Spatial structure alters from discrete microjets interacting with the stagnant fluid upon their discharge from the micronozzles, jet-to-jet interactions associated with their broadening as they decelerate downstream from the nozzles and exchange momentum with the relatively quiescent medium within the culture chamber 20, to stagnation/impingment flow with concomitant development of decelerating wall jets (along the FEP membrane 21 or a target surface) subject to biased discharge through peripherally located microchannel exits 37.

Close to the perfusing substrate 38 shown in FIG. 9a, flow is characterized by semi-confined, submerged, laminar impinging microjets issuing from an array of about 570 micronozzles (formed at the perfusing substrate 38) at moderate target-to-nozzle spacing. Upon their discharge from the nozzles, normal to the perfusing substrate 38, microjets interact with the stagnant medium within the culture chamber 20 as demonstrated in the sequence of images in FIGS. 9b-d normal to the jet axe. This shear driven interaction enables them to spread out. Measured spanwise velocity distributions reveal that the free boundary of microjets broaden with elevation, which is manifested by higher magnitudes of the induced velocity in the x-y planes, parallel to the perfusing substrate 38, as jets decelerate streamwise (along their axe) and accelerate in transverse planes. Momentum exchange between the jets and the stagnant medium causes this broadening with concomitant reduction in jet axial (streamwise) velocity. Before the jet interactions begin flow symmetry is determined by the number of microjets, with no apparent preferential direction as is shown in FIG. 9d.

With increasing distance from the micronozzles, formed at the perfusing substrate 38, discrete, submerged jets, located near the center of the culture chamber 20, whose radial spreading (normal to the jet axe) at first intensified with the elevation now begin to decelerate in the transverse planes, as is shown in FIG. 9e, parallel to the perfusing substrate 38. This can be seen in FIGS. 9d-e through a reduction in the magnitude of the radial microjet velocity at higher distances from the perfusing substrate 38. Initially accelerating radial outflows (closer to the micronozzles) eventually begin to decay due to interaction with the zero momentum medium within the culture chamber 20 and the presence of spatial constraints imposed by surrounding jets and peripheral confinement (cylindrical enclosure 35 surrounding the cultured domain).

As jets penetrate further into the culture chamber 20, the dynamics of the flow changes to incorporate the influence of the target surface and peripherally located microchannel exits 37. Further reduction in jet streamwise momentum causes their turning from the nominally vertical trajectory and adjoining microjets begin to interact prior to their impingement on the surface of the FEP membrane 21, FIGS. 9f-i. Jet interference prior to impingement is likely to be enhanced when jets are closely spaced and when the distance between the nozzle and the impingement plate (nozzle-to-plate distance) is relatively large. In the presently disclosed flow configuration, this causes the outflows from a number of microjets to merge, FIGS. 9f and 9g, and, begin vectoring towards the perimeter of the cultured domain, FIGS. 9h and 9i, at elevations that are lower than that of the microchannel exits 37 that start at z=350 μm. This vectoring is influenced by the interaction of the induced wall jet (developing along the FEP membrane 21) with adjacent impinging microjets. Although the spatial arrangement of micronozzles in the array generally determines the way the microjets interact with each other, a strong wall jet originating from the centrally located stagnation zone, FIGS. 9j-l, governs the jet-to-jet interactions with increasing distances from the perfusing substrate.

While in single jet impingement, fully developed wall jet ultimately decays with increasing distance from the stagnation point due to its radial spreading and rising thickness, (see for example Deshpande M. D. et al. in 1982 J Fluid Mech 114:213-236), the horizontal acceleration of a developing, centrally located, wall jet here, continues longer due to merging of outflows from a number of microjets. In fact, this strong wall jet deflects the axe of microjets located further away from the center of the culture chamber 20 and their impingement is delayed or even prevented. The induced, biased convection towards the microchannel exits 37 is more pronounced in microjets located closer to the cylindrical enclosure 35 than those located closer to the center of the culture chamber 20. Therefore jets emanating closer to the center of the culture chamber 20 penetrate deeper into the cultured domain than those issuing closer to the enclosure walls 35, with the microjet axial flow trajectory reduction from the center towards the perimeter. While conventional culturing of brain slices in-vitro obtains poor long-term viability at the center of the slice, realized flow field in the present arrangement facilitates the control of cellular microenvironment by providing ample amounts of media to the most vulnerable part of the tissue located at the center of the culture chamber 20 with the nutrient concentration reducing towards the lateral sides. Overall, with increasing radial coordinate, the axial transport reduces and lateral transport increases within the cell culture life-support chamber 20, with significant convective enhancement in stagnation and wall jet regions over passive diffusion transport mechanism.

While single impinging jet exhibits three characteristic flow domains consisting of a free jet (not influenced by the target surface), stagnation and a wall jet region, the closely spaced array of impinging jets herein obtains a single, centrally located stagnation zone due to interactions among adjoining wall jets and the topology of exit flow. The sequence of images in FIGS. 9(j-l) confirm the merging of microjet outflows to yield a stagnation point near the center of the FEP membrane aerator 21, with the magnitude of the induced velocity (parallel to the perfusing substrate 38) reducing with the proximity to the impingement plane where it reaches zero. Miniature imperfections in the fabrication process influence the symmetry of the flow and enhance the fluid turning towards the part of the culture chamber 20 where the flow resistance is lower. In the present experiments, the presence of a center mark on the perfusing substrate 38 alters the location of the nominally centrally located stagnation region, so that it appears slightly off-center.

Measurements confirm that microjets issuing closer to the center of the perfusing substrate 38 penetrate deeper into the culture chamber 20 specifically targeting the most starved part of the tissue, while jet interactions and peripheral withdrawal of perfusate aid in the establishment of a complex 3-D flow within the culture chamber 20.

### Temperature Measurements

The perfusion chamber 30 with the centrally forming culture chamber 20 is placed inside a cm-scale incubator 10 comprising a polystyrene enclosure 11 using nylon inserts such that biocompatible fluidic couplings pass through the bottom of the enclosure 11. The heated enclosure 11, which may have exemplary outer dimensions of 50×25×18 mm, contains a prescribed concentration of gases and maintains the temperature of the media and gases at about 37° C. The mini-incubator 10 contains a thermofoil heater 16 that heats the air inside the incubator 10. Gases necessary for cellular metabolism enter the incubator 10 through its inlet port 12 (forced by a mini-blower, for example, outside the incubator 10) and leave through the outlet port 13. Externally located and controlled mini blowers 19 force the appropriate gas mixture (depending on the cultured tissue) over the heater 16 located on one side of the enclosure 11. In this configuration, gases blow directly onto the surface of the heater 16, and are predominantly heated by forced convection inside the mini-incubator 10. Heated gas warms the perfusion chamber 30 located downstream from the heater 16. The surface of perfusion chamber 30 and the culture chamber 20 reaches the thermal equilibrium with surrounding gas within the heated enclosure 11 so that the nutrient medium entering the incubator 10 through fluidic coupling inserted into media inlet 33 (FIGS. 4 and 5), warms up to the prescribed temperature, during the slow media advance towards the cell culture chamber 20.

In operation, the perfusion chamber 30 remains fairly isothermal so that the fresh nutrient medium injected into the culture chamber 20 through the perfusion substrate 38 reaches the desired temperature 37±0.2° C. within its fluidic inlet port 33 well before entering the cultured volume, with the heated length increasing linearly with the flow rate. For typical flow rates used in 3-D cell culturing the required heated length for the nutrient medium to reach desired temperature within the perfusion chamber 30 is sub-millimeter scale, i.e., 1 mm fluidic path through the inlet port 33, for example, is enough to warm the injected medium to a prescribed temperature. The temperature of the injected media just underneath the perfusion substrate 38 may be measured using a thermocouple that is connected to the temperature controller 18 to maintain the temperature in the range of 36.8-37.2° C. The PID controller 18 may be coupled to a DC switching solid-state relay to regulate the heat flux dissipated by the heater 16. Owing to its 7 μl of volume, culture chamber 20 and the entire perfusion chamber 30 facilitate temperature control due to relaxed working conditions and low time constants with negligible delays before parameters reach desired values. Small characteristic dimensions of the centimeter-scale incubator 10 result in negligible losses to the ambient. For example, for the heated enclosure 11 to be maintained at 37° C. heater 16 needs to dissipate about 1 W (based on losses to the ambient at 20° C.). Likewise, a fraction of a milliWatt is needed to warm the media to 37° C. at the volume flow rate of 7 μl/min (about 1 exchange/minute) with sub-millimeter scale heated length through the perfusion chamber 30.

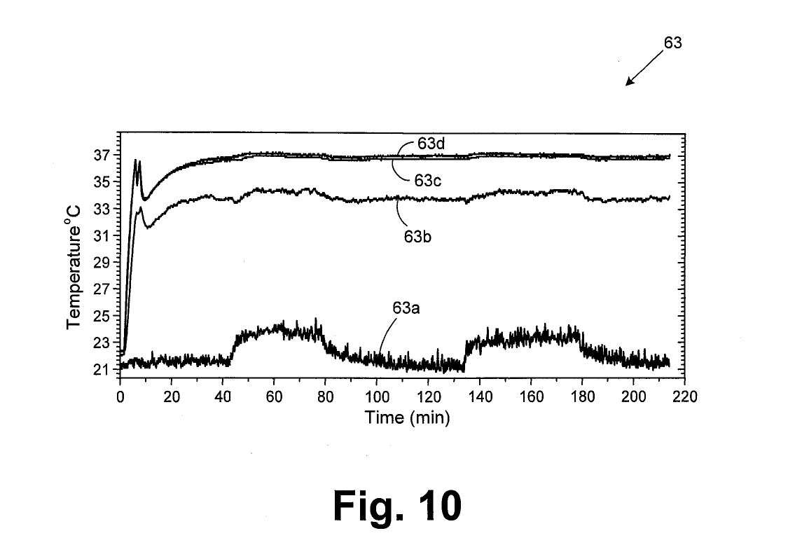

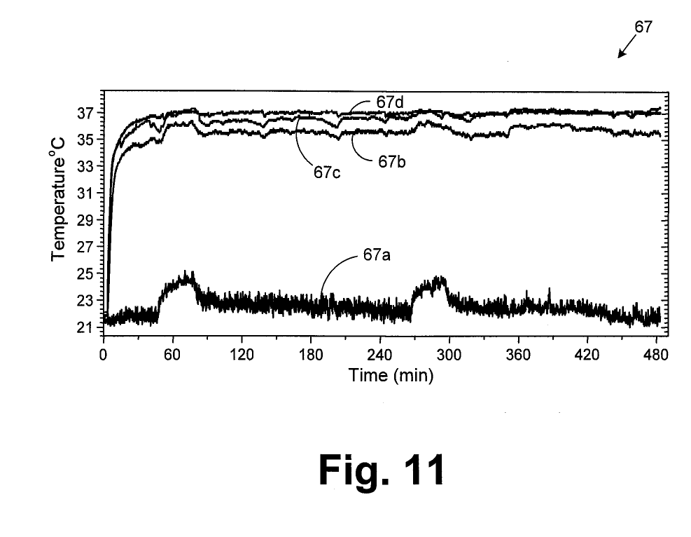

Temperature measurements inside the mini-incubator 10 may be taken at four locations, for example, using Fluke Hydra data acquisition unit with better than 0.1° C. temperature resolution. The temperature of the fresh nutrient medium is measured just before its injection into the culture chamber 20 (a thermocouple is placed immediately adjacent to the bottom side of the perfusion substrate 38 and routed through the media inlet port 33 via inlet tubing 24 and a small tee, and sealed). The temperature of the air inside the return flow collecting pool 41 may be measured by a thermocouple routed through the media outlet port 34. The temperature of air above the culture chamber 20 (i.e., above the FEP membrane 21) is also measured with the thermocouple routed through the side of the optically clear enclosure 11. The ambient air temperature outside the mini-incubator 10 is also measured. FIG. 10 shows graph 63 that illustrate thermocouple temperature measurements following an initial 10-minute long controller auto-tuning. The graph 63 sets forth measurements for ambient air 63a, air above the FEP membrane 63b, air inside the collecting pool 63c, and water just underneath the grid 63d. FIG. 11 shows graph 67 that illustrates temperature measurements of ambient air 67a, air above the FEP membrane 67b, air inside the collecting pool 67c, and water just underneath the grid 67d while the incubator is in operation.

For the PID controller 18 to maintain the temperature between a low and a high set point (37±0.2° C.), an auto-tune is performed during which an optimal set of constants (proportional, integral and differential) is found based on the thermal response of the system. After auto-tuning is finished, a low set point temperature is reached after about 45 minutes, as shown in FIG. 10. In operation, the controller 18 utilizes this set of parameters to maintain the temperature within a prescribed range in response to changes of ambient air temperature. Measurements taken during a 3-hour period, for example, demonstrate that the controller maintains the temperature of the media just before injection into the culture chamber 20 within the prescribed limits.

Measurements taken with a given set of parameters a few days later, as is shown in FIG. 11, demonstrate that after the initial warm-up period of the system, the temperature of the media remains within the set range over an 8-hour period during which measurements are taken. Thermocouple measurements reveal that the controlled media temperature just underneath the perfusion substrate 38 follows the temperature of the air inside the outer, collecting pool 41 where used media leaves the perfusion chamber 30.

A more uniform temperature distribution throughout the volume of the mini-incubator 10 may be easily obtained by insulating the exterior walls with a layer of foam-like tape, for example. This is also advantageous in preventing the visible light from entering the culture chamber 20 and disrupting the cells unless optical measurements are under way.

Multiple perfusion chambers 30 may be integrated within a single enclosure 11, for example, each with its own independent temperature controllers, or using the same controller, and gas sources. Several syringes can be used on each side of the syringe pump 19a moving block such that each perfusion chamber has an independent set of syringes for the simultaneous infusion of the culture medium and the withdrawal of catabolic waste products. This permits diagnostic testing of multiple samples in parallel without the possibility of cross contamination as perfusion chambers 30 are in fact microbe impermeable on the gas side. The volume flow rate of injected media can be different between adjacent perfusion chambers 30 by use of different pairs of syringes for particular chambers. For example, one culture chamber 20 can have exchange rate as much as 4 times higher, or more, than the other by choosing a syringe pair whose plungers have the surface area 4 times the surface area of plungers in another pair of syringes.

The scale-down from standard (refrigerator size) incubators to a cm-scale, portable, integrated, diagnostics incubator 10 obtains a remarkable physical enhancements such as the efficiency in heating or cooling the enclosures as they are miniaturized. This facilitates temperature control as heated or cooled centimeter-scale incubator obtains faster response times to temperature changes due to negligible inertia of an inherently miniature device, and, reduces costs associated with power consumption.

### Fabrication Methodology

Biocompatible and autoclavable, microfluidic perfusion chambers 30 may be rapidly prototyped using a process methodology that combines solid object printing and polymer replica molding (commonly referred to as the soft lithography, see for example Xia Y. and Whiteside G. M. in 1998 Angewandte Chemie 37:550-575). Custom or commercially available perfusion/cell attachment substrates are then sealed to the bottom of the cultured domain with a PDMS prepolymer, and cured.

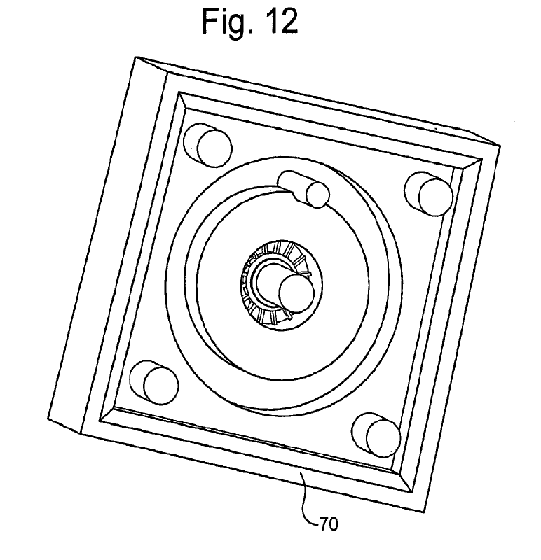

One example of a disposable master 70 for the elastomeric molding, as is shown in FIG. 12, are produced by solid-object printing (SOP) of an organic thermoplastic (wax-like) polymer that solidifies upon extrusion based on a defined 3-D CAD mold, for example. This convenient inkjet printing tool eliminates the need for expensive cleanroom facilities, and, unlike inherently planar 2-D photolithographic techniques, obtains truly 3-D objects whose feature resolution is 86 μm. Following the mold design phase, production is virtually hands free as printer fabricates the masters, layer by layer, directly onto a platform without the use of masks. In a few hours over fifty wax molds may be fabricated in a single print. The resolution of the commercially available Thermojet printer (3D Systems, Valencia, Calif.) used herein is 300×400×600 dpi, i.e. 85, 64 and 24 μm in x, y, and, z respectively. For the majority of microvascular systems used in bioengineering, this yields sufficient feature details and permits quick completion of several design iterations, fabrication and testing at a low cost. The smallest extruded features of the perfusion chamber mold is a positive relief for the elastomeric molding of 150 μm wide and 350 μm deep microchannels that are reproduced faithfully owing to the excellent shape conforming properties of the PDMS prepolymer.

Upon extrusion of the perfusion chamber molds, a 10:1 mixture of the PDMS pre-polymer base and cross-linking agent (SYLGARD 184 Silicone Elastomer Kit, Dow Corning, Midland, Mich.) is poured into the wax template, cured at room temperature for 48 hours, and, peeled off. The use of mold release agents was deemed unnecessary owing to favorable surface chemistries of materials in contact. However, to facilitate the release of cured polymer replicas from deep disposable molds, the top of their sidewalls are fabricated at a 45° angle. This ensures that the upper most layer of the polymeric PDMS material is entirely peeled-off, thus allowing good grip in stripping subsequent layers. During detachment, vertical columns that correspond to fluidic connections in the finished perfusion chamber 30 usually break from the mold base so that thermoplastic templates cannot be reused. Posts that remain within released replicas are simply pushed through the cast openings within PDMS using a thick needle. Depending on size, objects entrapped within released elastomer can be removed by either heating the replicas to melt the thermoplastic material followed by suction of the latter, or, in case of truly micron scale features by dipping them in organic solvents (e.g. OS-20 low molecular weight siloxane, Dow Corning, Midland, Mich.), thus causing the temporary swelling of PDMS and the release of trapped components.

To further reduce the prototyping costs, disposable, thermoplastic masters can be replaced by reusable, polymeric, master molds. Reusable templates can be fabricated using a “sandwich method”, i.e., by encapsulating the prepolymer within an extruded box, and, by inserting the cast PDMS perfusion chamber 30 into the box face down so that its base acts as a box lid. This enclosure box may be extruded using a 3-D printer such that the model interior dimensions correspond to the outer dimensions of the PDMS perfusion chamber 30. During insertion of the PDMS perfusion chamber 30 into the box, the excess prepolymer leaves the mold through fluidic openings located on the base of the perfusion chamber 30 (box lid). The procedure is straightforward and it takes only two sacrificial thermoplastic molds to fabricate a single reusable PDMS master.

### Favorable PDMS Material Properties

Polydimethylsiloxane (PDMS) is a rubbery, biologically compatible, microfabrication compatible, silicone elastomer that is low-cost, and generally non-toxic, non-flammable, thermally stable, chemically inert, optically clear, permeable to gases including oxygen and carbon dioxide and almost impermeable to water, see for example, Ng J. M. K. et al. in 2002 Electrophoresis 23:3461-3473. It conforms to submicron features with no discernible pattern degradation or surface distortion upon common biological handling and represents the material of choice for versatile biological and medical applications (see for example, McDonald J. C. et al. in 2002 Acc Chem Res 35:491-499; or Whitesides G. M., et al. in 2001 Ann Rev Biomed Eng 3:335-373). PDMS properties offer substantial advantages over fabrication methods in hard materials. Molding of 3-D structures in PDMS is inexpensive, easy, and a versatile method of making complex geometries. The sealing of fluidic devices, coupling of components, packaging and interfacing of integrated systems is fairly straightforward to employ in PDMS owing to its elasticity, watertight sealing properties and low electrical conductivity.

The modular design of the perfusion chamber 30 enables the use of commercially available or custom made substrates, thus introducing specific topography, flow characteristics and surface chemistry intended for particular set of experiments. Besides gold, PDMS, SU-8 photopolymer, polymethyl methacrylate (PMMA), and other materials may be used as perfusion/growth substrates upon certain surface treatments that render the surfaces cytophilic. Specifically, the ease of PDMS topographical and chemical patterning makes it an ideal candidate for perfusion substrate whose shape, texture and surface chemistry can be adjusted to promote cellular adhesion. Pulsed plasma deposition of allylamine on hydrophobic polydimethyl-siloxane (PDMS), for example, creates cytophilic cell adhesion cites that can be sterilized using steam autoclaving and re-used for several cycles (Harsch A, et al. in 2000 J Neurosci Meth 98:135-144). Attachment and growth of cultured neurons can be manipulated using microcontact printing methods that permit the deposition of thin layers of various organic materials on PDMS with varying degree of organized architecture. Selective patterning of PDMS substrates may also be necessary to confine cell growth to particular regions within the culture chamber 20. Silanizing oxidized PDMS with an amino-terminated silane (aminopropyltriethoxysilate) provides a reactive surface for a bifunctional cross-linker for protein attachment (Bernard A., et al. in 2001 Nat Biotechnol 19:866-869). A similar technique can be employed to attach amino-terminated polyethylene glycol (PEG) to make the surface stable against surface rearrangement and hydrophilic for days in air, e.g., Donzel C., et al. in 2001 Adv Mater 13:1164-1168. Bio-fouling, a non-specific protein adsorption to surfaces such as the interiors of microbore tubes or microchannels, for example, represents a common problem in microfluidics as it eventually leads to device clogging. Grafting a poly(ethylene glycol)di-(triethoxy)silane onto an oxidized PDMS surface, the surface of PDMS becomes permanently hydrophilic with reduced bio-fouling properties, see for example Papra A. et al. in 2001 Langmuir 17: 4090-4095).

### Enabling Qualities and Empowering Functionalities of the Diagnostics Incubator with Integrated Perfusion

The disclosed instrumented, portable, centimeter-scale incubator 10 represents a striking departure from the standard culture environment posing virtually no limits for field applications with the convenience of being compact, and, flexibility in design and operation that allows its integration into a more robust totally integrated systems. Hence, advantages of the miniaturized incubator 10 include (1) reduced size of the functional device, (2) flexibility in design (3) minimized response times in pharmacological and biochemical analyses owing to microliter volume culture chamber 20 and negligible time-delays in reaching steady state operation, (4) low expenditures associated with the amount of used reagents, (5) reduced production of toxic waster products, (6) decreased requirements for power, (7) reliable and reproducible operation owing to relaxed operating conditions associated with superior control of physical and chemical parameters in small scale devices, (8) increased speed of analyses, (9) the ability to operate in parallel a number of perfusion chambers 30 onboard a single centimeter-scale incubator 10, (11) low-cost devices resulting from fabrication of all components onboard a single, disposable platform 30, (12) optical, electrical and fluidic accessibility, (13) facile fabrication, packaging, integration and interfacing, and, (14) portability. Disclosed perfusion chamber 30 enables forced convection intercellular nutrient delivery thus extending the longevity of otherwise nutrient and gas depleted 3-D cultures.

A reduced-to-practice mini-incubator 10 enables simultaneous optical, fluidic and electrical interfacing, and data acquisition, in a controlled environment prescribed by the temperature, concentration, and flow rate of gases, nutrients or other relevant substances. In an exemplary embodiment, the temperature of the media inside the incubator 10, just before injection into the culture chamber 20, is controlled at 37±0.2° C. Associated infrastructure can support a number of mini-incubators 10 with their separate perfusion chambers 30. In-situ diagnostic testing may be performed without disruption of cultured tissue within separate mini-incubators 10, thereby reducing the chances of contamination.

Growing needs to miniaturize and scale-down cell cultures and their microenvironments in vitro from multi-well screening plates to disposable cell assays for biochemical analyses for example, in an environment that preserves the culture viability and allows in situ monitoring of their physiology, small-scale diagnostic incubators with integrated perfusion become necessary to manipulate the cellular environment down to molecular or cell level. Although surface forces become dominant following the scale-down in size of conventional systems, the similarities in physical and chemical parameters are generally preserved resulting in analogous biological behavior. Following the scaled-down inertia of the system reduces leading to faster response times to chemical stimuli and physical changes. This, in turn, offers unprecedented control of biological processes due to negligible time delays before parameters reach desired, steady state values and gives an insight into a real time dynamics of cellular responses to stimuli. A reduction in characteristic length scale usually results in relaxed operating conditions for sensors and actuators, often eliminating excessive hardware and high power associated with large-scale systems. Hence, unlike scaling up, scaling down simplifies incubator operation and control thereby substantially lowering costs, allowing parallel processing of multiple samples, and provides higher accuracy and reproducibility of performed assay analyses.

Compared to conventional culture tools using Petri dishes or multi-well screening plates, the centimeter-scale incubator 10 with its microliter volume perfusion platform 30 offers greater control over the cellular microenvironment. Using relatively small number of cells at a high plating density, cellular response to rapid changes in bathing medium composition, application of drugs, or the changes in the extracellular ionic concentration governing the cellular excitability can be accurately controlled and easily altered. While relevant scientific studies have been hindered by diffusion limited supply of the nutritive substances and gases necessary for cellular metabolism, the perfusion chamber 30 exposes the cells to continuous flow of media with rapid exchange rates to ensure plethora of nutrients and prevent the metabolic culture decay. Thus, in the centimeter-scale incubator 10, 3-D cultures can be easily maintained in vitro over extended periods of time with sufficient concentration of nutrients and gases at an optimal value of acidity and temperature so that the humoral states are not altered during the course of an experiment.

Concurrent, long-term, bio-culturing with in-situ diagnostics and sample preservation has numerous benefits. Controllable field culturing (away from major laboratories), tissue growth and analysis on a microscope stand, controllable injection of media and trophic factors, biomonitoring and biosensing are just a few applications where the mini-incubator 10 with its integrated perfusion chamber(s) 30, optical and electrical accessibility are desirable. While perfusion chambers with and without environmental control are available, e.g., neuronal perfusion, the utility and widespread use of these devices is often hindered by metabolically inadequate capillary action nutrient delivery, and, their inadaptability to be modular and address a wide range of tasks at a reasonable price. The design of the biocompatible perfusion chamber 30 ensures forced convection closed-loop continuous infusion of nutrients and withdrawal of waste with enhanced exposure to gases necessary for cell metabolism to extend the culture longevity beyond that achieved in large-scale incubators or small incubation baths. Disposable or steam autoclavable perfusion chamber 30 can be easily re-configured to meet a particular demand with a new device in hand in a matter of hours without compromising its simplicity.

The modular and low-cost design of mini-incubator 10 facilitates plug-in functionality of various building blocks that can be easily integrated (e.g., mixing chambers, nano-volume multi-port controllable injectors, microelectrode arrays, sensors, etc.) and allows operation either in full-scale laboratories or in the field, providing a portable, fully self-contained biological workstation. For example, perfusion chamber 30 may enable temporal and spatial control of injected agents in addition to continuous delivery of the cell culture media, Customized sample holders can be fitted as necessary with fluidic and electrical connectivity to facilitate in situ sample manipulation and diagnostics with negligible risk of contamination. The modular infrastructure can support a number of mini-incubators 10 with their separate perfusion chambers 30 that can be easily plugged in and out of a centralized system, providing maximum flexibility in experimental design and minimal capital expenditure. In-situ diagnostic testing may be performed within separate incubators 10, eliminating disruption of other cultured tissue and thereby dramatically reducing the chances of contamination. Various workstation designs may be constructed and inexpensively customized to allow for concurrent optical, fluidic and electrical interfacing to permit full experimental control, including data acquisition and individual sample preservation.

Thus, in summary, the incubator workstation 10 has optical, environmental and fluidic accessibility and enables simultaneous culturing and analyses on a microscope stand. Integrated, actively controlled, perfusion chamber 30 utilizes forced convection for intercellular nutrient supply and gas exchange thereby allowing the studies of thicker tissue slices and dissociated, high-density, three-dimensional (3-D) cultures over extended periods of time. Miniature cell culture life support chamber 20 (about 7 μl) offers relaxed operating conditions with superior environmental control and reduced expenditures associated with harvesting, culture preparation, the amount of used reagents and power consumption. A number of perfusion chambers 30 can be integrated onto a common platform for high-throughput culture screening with reduced risk of contamination. Superior culture aeration is achieved two-fold: (i) through an optically clear, microbe and water impermeable membrane 21 placed over the top of the culture chamber and via (ii) separate in-line venting bubble trap 26. A thin membrane 21 that encapsulates the perfusion chamber 30 enables efficient gas exchange and keeps the tissue moist in a sterile environment. It further reduces the risks of contamination by allowing the culturing in non-humidified incubators. The in-line aerator 26 ensures the equilibrium of the nutrient medium with the gas environment, via semi-permeable membrane 27, before being injected into the culture chamber 20. This device 26 also serves as an auto-venting bubble trap that prevents the bubbles from ever reaching the perfusion chamber 30. The use of in-line aerator 26 eliminates the need for a separate enclosure to equilibrate the media and a bubble trap to purge the bubbles, thus greatly simplifying the design and enabling development of cost-efficient compact devices. Spatially 42 or temporally varying concentrations of injected substances 41 into the culture chamber 20 can be dynamically controlled. Additional functionalities including but not limited to glucose consumption, pH or dissolved O₂ sensing and control 18a can be easily integrated into the device 30. Shape conforming and contact sealing properties of the polydimethylsiloxane polymer used in the fabrication of a perfusion chamber 30 enable straightforward fabrication of complex three-dimensional structures, sealing of devices, coupling of various microfluidic and electrical components, packaging and integration into functional systems that can be easily interfaced with the macro world, see for example Beebe D. J. et al. in 2000 Proc Natl Acad Sci USA 97: 13488-13493; Chabinyc M. L. et al. in 2001 Anal Chem 73: 4491-4498; or Zanzotto A. et al. in 2004 Biotechnol Bioeng 87:243-254.

The modular design with versatile plug-in-functionalities can be tailored to specific use and omitted in particular application. These functionalities include but are not limited to: microfluidics, e.g. micropumps, valves, mixers and microinjectors; introduction of spatially or temporally varying concentration gradients of injected substances into the culture chamber 20; the integration of microelectrode arrays for physiological studies; biosensors for detection and control of small molecules (oxygen, pH, glucose) and large molecules (immunosensors); the capabilities of cooling below the ambient temperature to preserve the tissue for example; or the packaging of a larger number of culture chambers onto a common platform.

Increasing demands for miniature, disposable cellular essays for long-term pharmacological or biochemical studies and tissue engineering require perfused, controllable cellular microenvironments. Controllable features include but are not limited to: the adjustable shape and texture of the cell culture life support chambers, specific surface chemistry or protein adsorption, and, tunable topography of extracellular matrices and chemically patterned microfabricated scaffolds that can only be realized in a microfabrication compatible, low-cost, mass produced, dynamic perfusion platforms. These requirements come in parallel with the needs to reduce the costs associated with the amount of used specimen, biochemicals, disposal of waste, excessive hardware associated with large-scale systems, power consumption, and, repeated analyses due to metabolic failures in the absence of functional vasculature and blood supply to the tissue upon harvesting.

Compared to conventional culture tools using Petri dishes or multi-well screening plates, the disposable or autoclavable culture chamber 20 measures about 7 μl in volume and offers superior control of the cellular microenvironment, see for example Vukasinovic J. et al. in 2006 Proceedings of the 2006 ASME Summer Bioengineering Conference. Owing to the facile fabrication methodology, straightforward integration and packaging, perfusion chamber 30 and the miniature incubator 10 can be easily modified to meet different requirements. For example, the ultimate challenge of diagnostic cell assays is to incorporate biosensors and control loops to monitor temperature, humidity, pH, oxygen or ionic concentration in a perfused system with adjustable, spatially selective, steady state or temporally varying, velocity or concentration gradients, all within a common platform that contains a large number of cell arrays ready for fundamental studies, optical diagnostics, and, intra- or extracellular stimulation and recording. Thus this constitutes one of the many applications where a centimeter-scale, diagnostics incubator 10 with the integrated perfusion chamber 30 can be used.

Recent interest in totally integrated systems should eventually result in development of inexpensive, microfabricated devices with automated dissociation, cellular manipulation and diagnostics, see for example Prokop A. et al. in 2004 Biomed Microdevices 6:325-339. These computer-controlled microsystems will incorporate perfusion chambers, incubators, sensors, laminar flow hoods and hardware related to cell feeding, counting, etc. on a single, seemingly disposable, platform. The disclosed centimeter-scale incubator 10 with integrated perfusion is an example of how conventional (refrigerator size) incubators can be scaled down, and how their utility can be empowered with fluidic functionality and optical accessibility. Other advantages of the scale-down include reduced response times to physical changes and chemical stimuli, higher repeatability of performed analyses, reduced number of cells at high plating densities, low cost with minimal volume requirements, and, the possibility for parallel processing of many samples onboard a single disposable platform.

As mentioned in the Background section, the dominant mode of mass transport in commercially available incubation baths remains diffusion and/or capillary action to passively augment the supply of media and oxygen to the tissue that eventually runs down metabolically. However, to meet and exceed the metabolic requirements for nutrient delivery and catabolic waste removal, mass transport by pure diffusion becomes insufficient and demands a convective enhancement for an adequate, dynamic, long-term control of the culture condition.

Such enhancement is achieved in the novel, perfusion chamber 30, where convection based intercellular mass transport can be actively controlled and problems associated with the flow perturbations at the interface (Haas chamber), for example, or the floating of the tissue and the injection of drugs (Zbicz chamber) completely eliminated. Recent experiments corroborate the utility of forced convection interstitial nutrient delivery obtained in the perfusion chamber setting 30. An efficient circulatory system disclosed herein facilitates the studies of thicker organotypic cortical brain slice cultures as demonstrated by Rambani K. et al. in 2006 at the Society for Neuroscience 36^{th} Annual Meeting, Neuroscience 2006. The integrated membrane 21 and an in-line aerator 26 enable gas transport from both sides of the brain slice.

The perfusion chamber 30 not only obtained high viability of perfused cortical slices, but also the perfusion of nutrient medium in larger scale chambers of a similar design proved to be a productive tissue engineering setting enabling the growth of high-density, physiologically more credible, 3-D tissue-equivalent constructs. Perfused neuronal-astrocytic co-cultures grown in an extracellular matrix at cell densities close to that of the brain tissue (10,000 cells/mm³) exhibited over 90% viability throughout their thickness (500-750 μm) while non-perfused, sister-cultures evidenced widespread death as described by Cullen D. K. et al. in 2006 at the 28^{TH} Annual International Conference IEEE Engineering Medicine and Biology Society, and by Cullen D. K. et al., 2007 J Neural Eng 4: 159-172. Note that culture models consisting of multiple cell types, such as neuronal/astrocytic co-cultures, for example, closer approximate the heterogeneity of the in vivo tissue. In particular, the heterogeneity of the nervous system is better represented through the physical support and the metabolic coupling between neurons and astrocytes, e.g. Tsacopoulos M. et al. in 1996 J Neurosci 16:877-885, or, Aschner M. in 2000 Neurotoxicology 21:1101-1107.

One of the empowering aspects of the biocompatible perfusion chamber 30 integrated within a centimeter-scale diagnostic incubator 10 lies in its straightforward adaptability to various application challenges by incorporating versatile microfluidics and MEMS functionalities with superior control of pertinent cellular microenvironment than that achieved in standard, multi-well screening plates, Petri dishes, or Haas- or Zbicz-top incubation baths. Other enabling qualities include the ability to integrate multielectrode arrays or biosensors to monitor physiological functions down to cellular level, and, generate complex, steady state e.g. Dertinger S. K. W. et al. in 2001 Anal Chem 73:1240-1246, or, temporally varying gradients of biologically active substances using microfluidic networks and different modes of perfusion. By actively controlling perfusion, the nature of cell feedings can be radically changed to enhance haptotaxis for example, e.g. axonal outgrowth and path finding along gradients of diffusible substances containing chemoattractants or repellents, e.g. Baier H. et al. in 1991 Science 255:472-475. Perfusion can be continuous, cyclic in a periodic or an aperiodic fashion following a specific control curve, push/pull, and could involve the re-use of the spent media for subsequent feedings in cases where high-exchange rates may lead to depletion of trophic factors secreted by the cells to support their own growth. Furthermore the dynamic perfusion system 30 facilitates the control over the composition of the bathing medium, its temperature, pH and the degree of oxygenation. Using the optical isolation of a pocket-size incubator 10, non-invasive sensing methods may be employed in measuring the amount of dissolved oxygen and pH using either the commercially available sensor foils, or lifetime fluorescence of oxygen and pH-sensitive dyes. In either case the sensing infrastructure remains outside the incubator enclosure, thus keeping it inexpensive, facile to fabricate, and, therefore disposable. The centimeter-scale integrated diagnostics incubator 10 is thus portable. Relevant hardware (pump, fan, controllers) may be battery operated and the appropriate gas mixture supplied in a small bottle. The same incubator 10 can be used to preserve biological specimen at a temperature that is below the ambient temperature by replacing the resistive heater with a miniature thermoelectric heat pump.

Portable, optically accessible, centimeter-scale, integrated diagnostics incubator for use in biological culturing with actively controlled forced convection perfusion has been disclosed. It is to be understood that the above-described embodiments are merely illustrative of some of the many specific embodiments that represent applications of the principles discussed above. Clearly, numerous other arrangements can be readily devised by those skilled in the art without departing from the scope of the invention.

1. A perfusion apparatus, comprising:
a perfused sample chamber;
at least one injection site disposed in an interior surface of the perfused sample chamber; and
the interior surface around the injection site is cytophillic so as to promote direct cellular attachment to the interior surface around the injection site.

2. The perfusion apparatus of claim 1, wherein the interior surface further comprises a perfusing substrate.

3. The perfusion apparatus of claim 2, wherein a plurality of injection sites are formed in the perfusing substrate.

4. The perfusion apparatus of claim 1, further comprising an optically transparent semi-permeable membrane enclosing a side of the perfused sample chamber.

5. The perfusion apparatus of claim 1, further comprising:
at least one inlet port in fluid communication with the at least one injection site; and
at least one outlet port.

6. The perfusion apparatus of claim 1, wherein the perfused sample chamber is steam autoclavable.

7. A perfusion arrangement, comprising:
a perfused sample chamber;
at least one injection site disposed in an interior surface of the perfused sample chamber;
a sample disposed in the perfused sample chamber;
the sample attached to the interior surface of the perfused sample chamber;
an agent flowing through the at least one injection site into the sample; and
the agent being forced through the sample by way of forced interstitial convection.

8. The perfusion arrangement of claim 7, wherein the interior surface of the perfused sample chamber further comprises a perfusing substrate.

9. The perfusion arrangement of claim 8, wherein a plurality of injection sites are formed in the perfusing substrate.

10. The perfusion arrangement of claim 7, further comprising an optically transparent semi-permeable membrane enclosing a side of the perfused sample chamber.

11. The perfusion arrangement of claim 7, further comprising:
an agent inlet in fluid communication with the at least one injection site; and
at least one outlet port.

12. The perfusion arrangement of claim 7, further comprising a control system configured to control a flow of the agent into the at least one injection site to create a temporally varying concentration gradient in the sample.

13. The perfusion arrangement of claim 7, further comprising a control system configured to control a flow of the agent into the at least one injection site to create a spatially varying concentration gradient in the sample.

14. The perfusion arrangement of claim 7, further comprising:
the at least one injection site further comprising a plurality of injection sites;
each of the injection sites being positioned with respect to a corresponding region in the perfused sample; and
each of the injection sites being in fluid communication with a corresponding inlet port.

15. The perfusion arrangement of claim 14, wherein a plurality of selected agents can be discreetly applied to the regions through a respective one of the inlet ports.

16. A perfusion method, comprising the steps of:
disposing a sample in a perfused sample chamber;
injecting an agent into a perfused sample chamber; and
forcing the agent through the sample by way of forced interstitial convection.

17. The perfusion method of claim 16, wherein the agent is injected into the perfused sample chamber through at least one injection site disposed in an interior surface of the perfused sample chamber.

18. The perfusion method of claim 16, wherein the agent is injected into the perfused sample chamber through at least one injection site disposed in a perfusion substrate that forms an interior surface of the perfused sample chamber.

19. The perfusion method of claim 16, further comprising the step of viewing the sample through an optically transparent semi-permeable membrane enclosing a side of the perfused sample chamber.

20-26. (canceled)

27. An incubator apparatus, comprising:
an enclosure;
a perfused sample chamber disposed in the enclosure;
at least one injection site disposed in an interior surface of the perfused sample chamber; and
at least the interior surface around the at least one injection site is conditioned so as to promote attachment of a sample to at least the interior surface around the at least one injection site, the attachment of the sample facilitating a forcing of the agent through the sample by way of forced interstitial convection.

28. The incubator apparatus of claim 27, wherein the entire interior surface of the perfused sample chamber is conditioned so as to promote the attachment of the sample.

29. The incubator apparatus of claim 27, wherein the perfused sample chamber includes an integrated multi-electrode array.

30. The incubator apparatus of claim 27, wherein at least one portion of the enclosure is transparent to allow optical inspection of a sample within the perfused sample chamber disposed in the enclosure.

31. The incubator apparatus of claim 27, further comprising an optically transparent semi-permeable membrane enclosing a side of the perfused sample chamber.

32-38. (canceled)

39. An incubator apparatus, comprising:
an enclosure having a through-hole sealed with a transparent membrane facilitating optical inspection of an interior of the enclosure;
a perfused sample chamber disposed within the enclosure; and
an optically transparent, semi-permeable membrane enclosing a side of the perfused sample chamber.

40. The incubator apparatus of claim 39, wherein a microscope objective can be positioned directly adjacent to the perfused sample chamber, where the content of the perfused sample chamber can be viewed through the optically transparent, semi-permeable membrane and the transparent membrane.

41. The incubator apparatus of claim 39, wherein an exterior side of the transparent membrane can be covered by an optical medium.

42. The incubator apparatus of claim 39, wherein the enclosure is opaque.

43-44. (canceled)

45. A method for inspecting a sample in an incubator apparatus, comprising the steps of:
maintaining a sample in a perfused sample chamber disposed in an enclosure, where a side of the perfused sample chamber is enclosed by an optically transparent, semi-permeable membrane, and the enclosure includes a through-hole sealed with a transparent membrane; and
positioning an objective lens into the through-hole in the enclosure and adjacent to the sample in the perfused sample chamber.

46. The method of claim 45, further comprising the step of disposing an optical medium onto an exterior surface of the transparent membrane.

47. The method of claim 45, further comprising the step of pressing the objective lens against the transparent membrane, thereby causing the transparent membrane to stretch.

48-50. (canceled)
